(19) United States
(12) Patent Application Publication
(10) Pub. No.: US 2005/0182451 A1
(43) Pub. Date: Aug. 18, 2005

(54) Implantable device with improved radio frequency capabilities

(76) Inventors: Adam Griffin, Apex, NC (US); Sean Saint, San Diego, CA (US); Mark Brister, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/034,344

(22) Filed: Jan. 11, 2005

Related U.S. Application Data
(60) Provisional application No. 60/535,885, filed on Jan. 12, 2004.
(60) Provisional application No. 60/535,914, filed on Jan. 12, 2004.

(51) Int. Cl. A61N 1/375

(52) U.S. Cl. 607/36

(57) ABSTRACT

Systems and methods for implantable devices that transmit and receive RF transmissions are provided. More particularly, the implantable device includes an antenna encapsulated within a non-hermetic material, wherein the antenna is spaced from the non-hermetic material by an air gap. Preferably, the spacing is provided by one or more enclosures that maintain the air gap surrounding the antenna during and after the manufacture of the device. The one or more enclosures can be in the form or tubing formed from glass, or the like, at least partially surrounding the antenna. By increasing the amount of air encapsulated within the implantable device, and particularly proximal to (e.g., around) the antenna, the susceptibility to changes in RF performance is reduced.

### RELATED APPLICATIONS

This application claims the benefit of U.S. Provisional Application No. 60/535,885 filed Jan. 12, 2004, and U.S. Provisional Application No. 60/535,914 filed Jan. 12, 2004, both of which are incorporated by reference herein in their entirety, and both of which are hereby made a part of this specification.

### FIELD OF THE INVENTION

The present invention relates generally to systems and methods for implantable devices that transmit and receive radio frequency transmissions.

### BACKGROUND OF THE INVENTION

A variety of implantable medical devices are known in the art for purposes such as sensors for diagnostic testing, blood pumps, pacemakers, and the like. Many of these devices transmit and receive information via Radio Frequency (RF) through or from a patient's body to a location remote therefrom. Some of these devices are formed from hermetic materials (e.g., titanium) in order to protect the sensitive RF components from the effects that can occur to an implanted medical device in vivo, for example, due to moisture penetration. Unfortunately, this design suffers from complexity of design and manufacture and/or higher density and mass than otherwise necessary.

### SUMMARY OF THE INVENTION

In a first embodiment, a device suitable for implantation in a body is provided, the device comprising an antenna encapsulated within a non-hermetic material, wherein the antenna is spaced from the non-hermetic material by an air gap.

In an aspect of the first embodiment, the air gap is maintained by an enclosure. The enclosure can surround at least a portion of the antenna. The antenna can be contained within at least a portion of the enclosure. The enclosure can comprise a hermetic material, such as glass. Alternatively, the enclosure can comprise a non-hermetic material, such as a polymeric material. The enclosure can comprise at least one tube, and the tube can comprise a wheel-like configuration.

In an aspect of the first embodiment, the air gap is maintained by at least one spacer that maintains a fixed distance between at least two support structures.

In an aspect of the first embodiment, the non-hermetic material comprises a plurality of hollow gas-filled beads.

In an aspect of the first embodiment, the device comprises a wholly implantable glucose sensor.

In an aspect of the first embodiment, the device comprises electronics, and wherein the non-hermetic material is molded around the electronics and antenna.

In a second embodiment, a method for forming a device suitable for implantation in a body is provided, the method comprising providing device electronics comprising an antenna configured for radiating or receiving an RF transmission, wherein the antenna is at least partially surrounded by an enclosure; and molding a non-hermetic material around the sensor electronics such that an air gap is maintained within the enclosure at least partially surrounding the antenna, whereby a device suitable for implantation is a body is obtained.

In an aspect of the second embodiment, the enclosure comprises a hermetic material, such as glass. The enclosure can comprise at least one glass tube.

In an aspect of the second embodiment, the device comprises a wholly implantable glucose sensor, and wherein the electronics are configured to process a signal from the glucose sensor.

### BRIEF DESCRIPTION OF THE DRAWINGS

FIG. 1A is a cross-sectional view through an implantable device having an antenna and provided with one or more enclosures containing a gas.

FIG. 1B is a cross-section through the antenna and tubes of the device of FIG. 1A.

FIG. 2 is a cross-sectional view of an antenna and a wheel-like tubing structure that provides for an air gap surrounding the antenna.

FIG. 3 is a cross-sectional view of an antenna and tubing structure wherein an antenna is held within spacers.

FIG. 4 is a perspective view of a continuous glucose sensor implanted within a human and a receiver for receiving data from the continuous glucose sensor via RF and subsequently processing and displaying glucose sensor data.

FIG. 5 is a perspective view of a continuous glucose sensor having a sensing region.

FIG. 6 is a block diagram that illustrates the electronics associated with an implantable glucose sensor.

FIG. 7 is a perspective view of the glucose sensor FIG. 5, showing sensor electronics in phantom.

FIG. 8 is a cross-sectional view through line 8-8 of FIG. 7.

### DETAILED DESCRIPTION OF THE PREFERRED EMBODIMENT

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

Definitions

In order to facilitate an understanding of the preferred embodiments, a number of terms are defined below.

The term “host,” as used herein, is a broad term and is used in its ordinary sense, including, but not limited to, mammals such as humans.

The term “non-hermetic material,” as used herein, is a broad term and is used in its ordinary sense, including, but not limited to, a material that allows the ingress of gasses and/or fluids. Non-hermetic materials include insulating materials, water-vapor permeable materials, and polymeric materials, such as epoxies, urethanes, silicones, Parylene, and the like.

The term “beads” as used herein, is a broad term and is used in its ordinary sense, including, without limitation, bubbles or other hollow or enclosed spaces filled with a gas, a vacuum, or low density material (wherein the density is compared to that of the enclosing or non-hermetic material).

The term “RF transceiver,” as used herein, is a broad term and is used in its ordinary sense, including, but not limited to, a radio frequency transmitter and/or receiver for transmitting and/or receiving signals.

The term “antenna,” as used herein, is a broad term and is used in its ordinary sense, including, but not limited to, a metallic or conductive device (such as a rod or wire) for radiating or receiving radio waves.

The terms “raw data stream” and “data stream,” as used herein, are broad terms and are used in their ordinary sense, including, but not limited to, an analog or digital signal directly related to the analyte concentration measured by the analyte sensor. In one example, the raw data stream is digital data in “counts” converted by an A/D converter from an analog signal (for example, voltage or amps) representative of an analyte concentration. The terms broadly encompass a plurality of time-spaced data points from a substantially continuous analyte sensor, which comprises individual measurements taken at time intervals ranging from fractions of a second up to, for example, 1, 2, 3, 4, or 5 minutes or longer.

The term “electronic circuitry,” as used herein, is a broad term and is used in its ordinary sense, including, but not limited to, the components (for example, hardware and/or software) of a device configured to process data. In a glucose sensor, the data includes biological information obtained by a sensor regarding the concentration of the analyte in a biological fluid.

The terms “operably connected” and “operably linked,” as used herein, are broad terms and are used in their ordinary sense, including, but not limited to, one or more components being linked to another component(s) in a manner that allows transmission of signals between the components. For example, one or more electrodes can be used to detect the amount of glucose in a sample and convert that information into a signal; the signal can then be transmitted to an electronic circuit. In this case, the electrode is “operably linked” to the electronic circuit. These terms are broad enough to include wired and wireless connectivity.

Overview

Implantable devices that are encapsulated in a non-hermetic material (e.g., epoxy), particularly wherein the non-hermetic material comes into direct or close contact with the antenna, typically transmit data via radio frequency. These devices typically use electrically-small antennas, which tend to have a high Q, making the antenna resonant frequency shift strongly depending on the environment (e.g., dielectric constant of the encapsulating material can shift over time as moisture penetrates through the encapsulating material and proximal to the antenna). Unfortunately, this shift in frequency response causes the efficiency of the antenna to change as it is encapsulated within an implantable device and implanted inside the body (e.g., due to moisture penetration through a moisture-permeable encapsulating material, such as epoxy). Therefore, it can be advantageous to improve the efficiency of the antenna by maintaining a substantially constant dielectric property of the device surrounding the antenna over time even when implanted in a host body.

Conventional prior art implantable sensors that have electronics therein generally use a hermetic material for at least a portion of the body that houses the sensitive RF electronics. However, conventional hermetic implantable devices suffer from numerous disadvantages including, for example, difficulty in RF transmissions through the hermetic material, seams that can allow water vapor penetration if not perfectly sealed, minimal design or shape changes without major manufacturing changes (inability to rapidly iterate on design), the need to mechanically hold and reinforce the electronics inside, and increased weight and density.

To overcome the disadvantages of the prior art, the preferred embodiments enclose the device electronics in a non-hermetic body. In one embodiment, the non-hermetic material is molded around the device electronics to form the body of the device. This configuration can offer a number of advantages, including, rapid design iterations (for example, changes in design geometry without mold changes), the ability to machine into precise dimensions and curvatures, enhanced RF transmissions, enhanced mechanical integrity of components (because, for example, the material fills around the electronics to form a monolithic piece and hold components in place), the ability to complete multiple cures (for example, to provide a seamless exterior), and reinforcement of fragile electrical components. In preferred embodiments, the material is epoxy; however other plastics can also be used, for example, silicone, urethane, and other non-hermetic materials, to name but a few. In some alternative embodiments, the device is formed from a non-hermetic shell and configured to receive the device electronics therein, which can provide some of the above-described advantages.

Selected embodiments provide an implantable device that includes encapsulated air within the device, preferably proximal to (e.g., around) the antenna. By increasing the amount of air encapsulated within the implantable device, the susceptibility to changes in RF performance is reduced by utilizing an air gap surrounding the antenna.

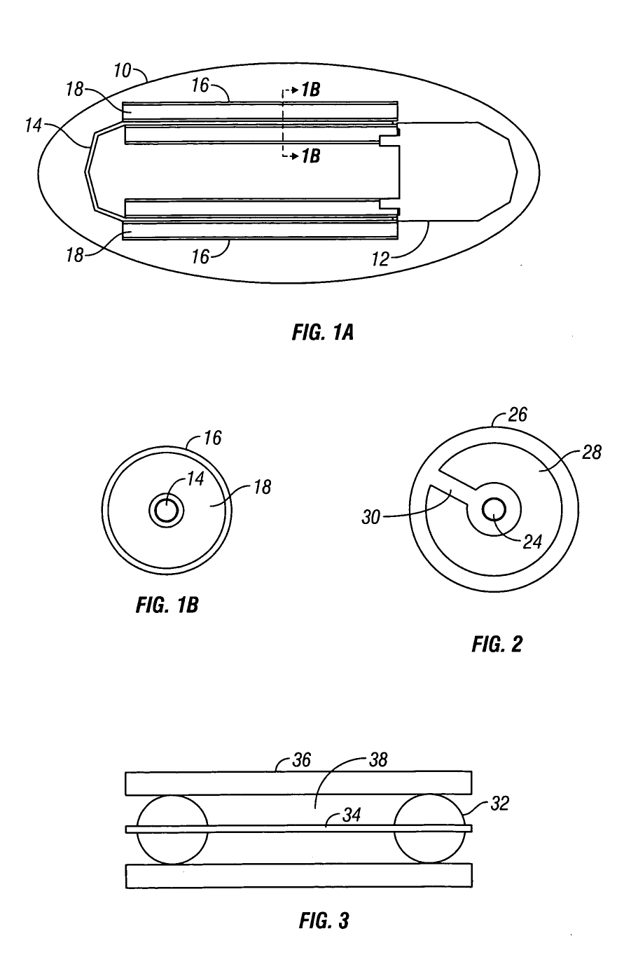

In a first embodiment, one or more enclosures are provided within the implantable device. The enclosure(s) contain vacuum or gas (e.g., air) therein. FIG. 1A illustrates one such embodiment.

FIG. 1A is a cross-sectional view through an implantable device showing the device body 10 including a circuit board 12 that supports the device electronics, an antenna 14 configured to radiate or receive RF transmissions, and a pair of tubes 16 configured to surround at least a portion of the antenna 14 for maintaining an air gap between the antenna and the device body 10. In some embodiments, the device body 10 is formed from a non-hermetic material and is configured to encapsulate the device electronics, including the circuit board 12 and antenna 14. In preferred embodiments, the device body 10 is molded or otherwise deposited around the electronics such as is described in co-pending U.S. patent application Ser. No. 10/838,912, filed May 3, 2004, and entitled, “IMPLANTABLE ANALYTE SENSOR.” However, in some alternative embodiments, the device body 10 is formed from a shell that is designed to enclose the device electronics therein, wherein the shell body is designed to include air surrounding the sensor electronics within the device.

The tubes 16, shown in cross-section in FIG. 1, surrounding at least a portion of the antenna 14 to maintain an air gap 18 around at least a portion thereof. FIG. 1B is a cross-section through the antenna 14 and tubes 16 of the device of FIG. 1A, showing the air gap 18 in another perspective. Namely, the tubes 16 are designed with a donut-like cross-section that enables positioning of the antenna 14 while maintaining the air gap 18 of the preferred embodiments. In this embodiment, the tubes are configured to hold a vacuum or a gas (e.g., air, nitrogen, argon, or the like) within a closed portion 18 of the tube and to allow the antenna 14 to extend through a center of the tubing. However, the tubes can be of any suitable cross-section and include an enclosure for holding a vacuum or gas and allow at least a portion of the antenna to extend therethrough. In some alternative embodiments, the tubes can be replaced with U-shaped, C-shaped, or spiral-shaped tubes so that the antenna can loop around more than once. In another alternative embodiment, the antenna can be totally encapsulated in tubing. In yet another alternative embodiment, the tubes include a wheel-like configuration with spokes holding the antenna in the center, such as described with reference to FIG. 2, below.

In preferred embodiments, the tubes 16 are formed from glass because of its inherent hermeticity. This hermeticity can be advantageous because water will not condense within the glass tubes over time. In some circumstances, water condensation can cause water drops to form and change the RF performance as described above. Additionally, water drops can add weight and/or density to the device, which can be sub-optimal in certain uses and applications, for example, an analyte sensor such as described in co-pending U.S. application Ser. No. 10/646,333, filed Aug. 22, 2003, entitled, “OPTIMIZED SENSOR GEOMETRY FOR AN IMPLANTABLE GLUCOSE SENSOR”, which is incorporated herein by reference in its entirety. However, in alternative embodiments, plastic (e.g., a Parylene coated plastic antenna holding tube) can be used when hermeticity within the implant is not a consideration.

FIG. 2 is a cross-sectional view of an antenna and tubing showing another embodiment that provides for an air gap surrounding the antenna. The antenna 24 is shown in cross-section surrounded by a wheel-like tubing structure 26 and air gap 28. In this embodiment, one-spoke 30 is shown on the wheel; however more spokes can be provided. This embodiment provides for improved RF capabilities for the same reasons described above, and further enables centering of the antenna for improved reliability.

FIG. 3 is a cross-sectional view of an antenna and tubing showing yet another alternative embodiment that provides for an air gap surrounding the antenna. In this embodiment, an antenna 34 is held within spacers 32. The spacers 32 can both to provide an encapsulated air gap 38 such as described above, and to position and maintain the antenna within the air gap provided by the tubing or other support structure 36 such as described in more detail above. For example, the antenna 34 is threaded through a through-hole within the spacers 32, which can be glass spheres, or the like. This embodiment provides sufficient air around the metal antenna, making it less sensitive to tuning changes when implanted inside the body.

In some alternative embodiments, other methods and configurations can be used to space the epoxy from the antenna, thereby providing an air gap therebetween. In one alternative embodiment, the antenna is encapsulated within a glass tube, and circumferential indentations are provided on the glass tube for centering and holding the antenna centrally therein. In yet another alternative embodiment, unused portions of the device (e.g., portions of the circuit board or other extraneous materials) can be substituted with glass beads or particles packed together, such as described in more detail with reference to co-pending U.S. patent application Ser. No. ______, filed on even date herewith, and entitled “COMPOSITE MATERIAL FOR AN IMPLANTABLE DEVICE.” Other configurations for glass and/or plastic spacing and air encapsulating devices are considered within the scope of the preferred embodiments.

In yet another alternative embodiment (not shown), small glass beads can be loaded into the non-hermetic material (e.g., epoxy) that forms the body of the implantable device (e.g., encapsulates the device) in order to introduce air around the antenna elements such as described in co-pending U.S. patent application Ser. No. ______, filed on even date herewith, and entitled, “COMPOSITE MATERIAL FOR IMPLANTABLE DEVICE”. The configuration of this embodiment can be used alone or in combination with the other embodiments described herein. These glass beads are preferably extremely small (e.g., smaller than 1/1000^{th} of an inch) and resemble talcum powder. Because these glass beads contain air, they provide improved RF performance and decreased density and over weight of the implantable device. Additionally, glass beads loaded within the implant can create neutrally buoyancy (e.g., density of 1 g/cc), which can be advantageous in some uses and applications such as described above.

While hollow, or air filled, glass beads are generally preferred, any suitable material of reduced dielectric content or reduced density, when compared to that of the insulating material (the epoxy) can be employed. For example, hollow epoxy beads, or hollow beads prepared from another material, such as a polymeric, ceramic, or metallic material, can also be employed. In addition to hollow beads, beads comprising an encapsulated open celled foam, or an encapsulated or unencapsulated closed cell foam can also be employed. For example, expandable polystyrene beads can be employed. In addition to beads, it is contemplated that the epoxy can be foamed such that air bubbles are defined within the epoxy material. While beads are generally preferred, any suitable shape can be employed, for example, cubes, rods, irregular shapes, and the like. While epoxy materials are generally preferred as the insulating material, any suitable material can be employed, for example, other polymeric materials, ceramics, metals, glasses, and the like, as will be appreciated by one skilled in the art.

The beads or other fill material can be of any suitable size. Preferably, the beads range in size from a few microns or smaller to a few millimeters or larger in their greatest dimension. Generally, filler having particle sizes of about 0.001, 0.005, 0.01, 0.05, 0.1, or 0.5 mm to about 1, 2, or 3 mm in greatest dimension are generally preferred. A variety of sizes and shapes of filler particles can be mixed together to improve the number of particles that can be packed into a certain volume. Other preferred embodiments employ an epoxy or other polymeric foams, wherein the voids are filled with a gas or vacuum.

Exemplary Continuous Glucose Sensor Configuration

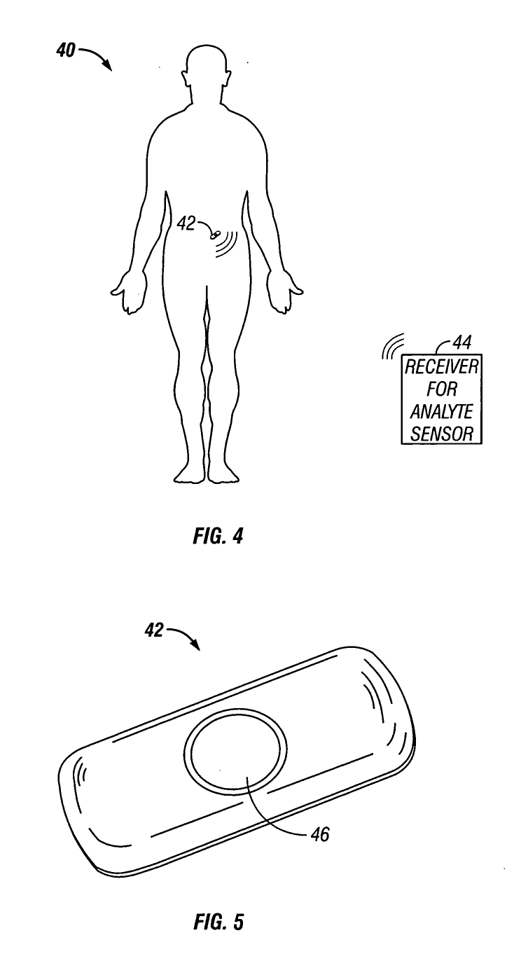

FIG. 4 is a perspective view of a system that utilizes the preferred embodiments, including a continuous glucose sensor 42 implanted within a human 40 and a receiver 44 for receiving data from the continuous glucose sensor 42 via RF and subsequently processing and displaying glucose sensor data. The system of the preferred embodiments provides improved wireless transmissions through the physiological environment, and thereby increases overall patient confidence, safety, and convenience.

The continuous glucose sensor 42 measures a concentration of glucose or a substance indicative of a concentration or a presence of glucose. However, the concepts described with reference to the sensor 42 can be implemented with any sensor capable of determining the level of any analyte in the body, for example oxygen, lactase, insulin, hormones, cholesterol, medicaments, viruses, or the like. Additionally, although much of the description of the glucose sensor is focused on electrochemical detection methods, the systems and methods can be applied to glucose sensors that utilize other measurement techniques, including enzymatic, chemical, physical, spectrophotometric, polarimetric, calorimetric, radiometric, or the like.

Reference is now made to FIG. 5, which is a perspective view of the implantable glucose sensor 42 of the preferred embodiments. Co-pending U.S. patent application Ser. No. 10/838,912, filed May 3, 2004, and entitled, “IMPLANTABLE ANALYTE SENSOR” and U.S. Patent Publication No. 2003/0032874 A1 disclose systems and methods that can be used with this exemplary glucose sensor embodiment. In this embodiment, a sensing region 46 is shown on the glucose sensor 42. In one preferred embodiment, the sensing region 46 comprises an electrode system including a platinum working electrode, a platinum counter electrode, and a silver/silver chloride reference electrode. However a variety of electrode materials and configurations can be used with the implantable glucose sensor of the preferred embodiments. The top ends of the electrodes are in contact with an electrolyte phase (not shown), which is a free-flowing fluid phase disposed between a sensing membrane and the electrodes. In one embodiment, the counter electrode is provided to balance the current generated by the species being measured at the working electrode. In some embodiments, the sensing membrane includes an enzyme, for example, glucose oxidase, and covers the electrolyte phase. In a glucose oxidase based glucose sensor, the species being measured at the working electrode is H₂O₂. Glucose oxidase catalyzes the conversion of oxygen and glucose to hydrogen peroxide and gluconate according to the following reaction:
Glucose+O₂→Gluconate+H₂O₂

The change in H₂O₂ can be monitored to determine glucose concentration, because for each glucose molecule metabolized, there is a proportional change in the product H₂O₂. Oxidation of H₂O₂ by the working electrode is balanced by reduction of ambient oxygen, enzyme generated H₂O₂, or other reducible species at the counter electrode. The H₂O₂ produced from the glucose oxidase reaction further reacts at the surface of working electrode and produces two protons (2H⁺), two electrons (2e⁻), and one oxygen molecule (O₂).

A potentiostat is employed to monitor the electrochemical reaction at the electroactive surface(s). The potentiostat applies a constant potential to the working and reference electrodes to determine a current value. The current that is produced at the working electrode (and flows through the circuitry to the counter electrode) is substantially proportional to the amount of H₂O₂ that diffuses to the working electrode. Accordingly, a raw signal can be produced that is representative of the concentration of glucose in the user's body, and therefore can be utilized to estimate a meaningful glucose value.

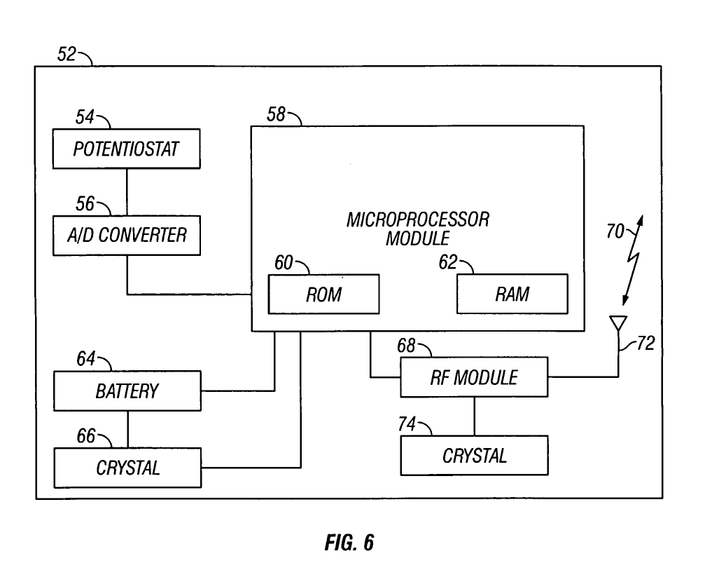

FIG. 6 is a block diagram that illustrates the electronics 52 associated with the implantable glucose sensor 42 in one embodiment. In this embodiment, a potentiostat 54 is shown, which is operably connected to an electrode system (such as described above) to obtain a current value, and includes a resistor (not shown) that translates the current into voltage. An A/D converter 56 digitizes the analog signal into “counts” for processing. Accordingly, the resulting raw data stream in counts is directly related to the current measured by the potentiostat 54.

A processor module 58 includes the central control unit that houses ROM 60 and RAM 62 and controls the processing of the sensor electronics 52. In some embodiments, the processor module includes a microprocessor, however a computer system other than a microprocessor can be used to process data as described herein, for example an application-specific integrated circuit (ASIC) can be used for some or all of the sensor's central processing, as is appreciated by one skilled in the art. The ROM 60 provides semi-permanent storage of data, for example, storing data such as sensor identifier (ID) and programming to process data streams (for example, programming for data smoothing and/or replacement of signal artifacts such as described in copending U.S. patent application Ser. No. 10/648,849, filed Aug. 22, 2003, and entitled, “SYSTEMS AND METHODS FOR REPLACING SIGNAL ARTIFACTS IN A GLUCOSE SENSOR DATA STREAM,” which is incorporated herein by reference in its entirety). The RAM 62 can be used for the system's cache memory, for example for temporarily storing recent sensor data. In some alternative embodiments, memory storage components comparable to ROM 60 and RAM 62 can be used instead of or in addition to the preferred hardware, such as dynamic-RAM, static-RAM, non-static RAM, EEPROM, rewritable ROMs, flash memory, or the like.

A battery 64 is operably connected to the sensor electronics 62 and provides the necessary power for the sensor. In one embodiment, the battery is a lithium manganese dioxide battery, however any appropriately sized and powered battery can be used (for example, AAA, nickel-cadmium, zinc-carbon, alkaline, lithium, nickel-metal hydride, lithium-ion, zinc-air, zinc-mercury oxide, silver-zinc, and/or hermetically-sealed). In some embodiments, the battery is rechargeable. In some embodiments, a plurality of batteries can be used to power the system. In yet other embodiments, the sensor can be transcutaneously powered via an inductive coupling, for example. In some embodiments, a quartz crystal 66 is operably connected to the processor 58 and maintains system time for the computer system as a whole.

An RF module 68 is operably connected to the microprocessor 58 and transmits the sensor data from the sensor to a receiver within a wireless transmission 70 via antenna 72. In some embodiments, a second quartz crystal 74 provides the system time for synchronizing the data transmissions from the RF transceiver. In some alternative embodiments, however, other mechanisms, such as optical, infrared radiation (IR), ultrasonic, or the like, can be used to transmit and/or receive data.

In the RF telemetry module of the preferred embodiments, the hardware and software are designed for low power requirements to increase the longevity of the device (for example, to enable a life of 3 to 24 months, or more) with maximum RF transmittance from the in vivo environment to the ex vivo environment (for example, a distance of from about one to ten meters or more). Preferably, a high frequency carrier signal in the range of 402 to 405 MHz is employed in order to maintain lower power requirements. Additionally, the carrier frequency is adapted for physiological attenuation levels, which is accomplished by tuning the RF module in a simulated in vivo environment to ensure RF functionality after implantation. Accordingly, the preferred glucose sensor can sustain sensor function for 3 months, 6 months, 12 months, or 24 months or more.

In one embodiment, the body of the sensor is preferably formed from epoxy molded around the sensor electronics; however in alternative embodiments, the body can be formed from a variety of non-hermetic materials enclosing or encapsulating the sensor electronics in a variety of manners. Co-pending U.S. patent application Ser. No. 10/838,909, filed May 3, 2004, and entitled, “IMPLANTABLE MEDICAL DEVICE,” which is incorporated herein by reference in its entirety, describes systems and methods for encapsulation of RF circuitry in a non-hermetic (e.g., water vapor permeable) material, such as epoxy. In one alternative embodiment, the body is formed from a shell that opens to receive the sensor electronics, which are designed to fit within the body of the shell.

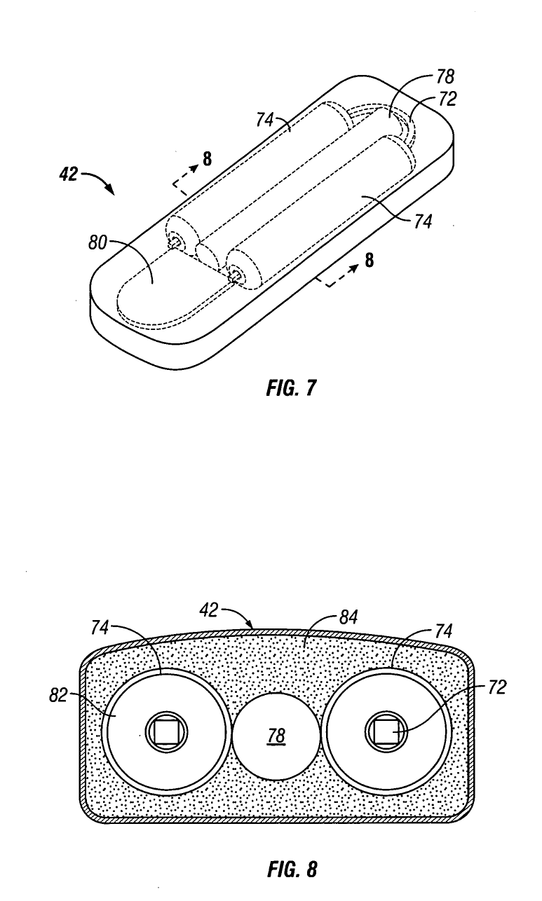

FIG. 7 is a perspective view of the exemplary glucose sensor 42 of FIG. 5, showing sensor electronics in phantom. In this embodiment, the glucose sensor 42 includes an antenna 72 for radiating and receiving RF transmissions and surrounded by one or more tubes 74 that maintain an air gap (see FIG. 8) proximal to the antenna 72. A battery 78 and circuit board 80 are shown that support the various components of sensor electronics such as described in more detail with reference to FIG. 6. However, the battery and other sensor electronics may be modified, moved, or removed as is appreciated by one skilled in the art.

FIG. 8 is a cross-sectional view through line 8-8 of FIG. 7. Particularly, the cross-section shows the donut-like configuration of the tubes 74. Because the tubes 74 are closed at their ends, air 82 (or other gas or vacuum) is maintained inside the tubes. The antenna 72 is threaded through the center of the tubes and can be designed to fit with such a tolerance as to allow minimal to no space between the tubes 74 and the antenna 72. In this embodiment, a non-hermetic material 84 encapsulates the device around the sensor electronics to form the body of the sensor as described in more detail above. It is noted that even when some spacing exists between the tubes 74 and the antenna 72, the non-hermetic material is designed with such a viscosity such that it cannot easily penetrate through the spacing during the molding process. Alternatively, even if the non-hermetic material were to penetrate through the spacing between the antenna 72 and the tubes 74, the air gap 82 provided by the tubes 74 is in such proximity to the antenna to achieve the benefits described in the preferred embodiments.

In the illustrated embodiment of FIGS. 7 and 8, tubes 74 are enclosed to maintain air or other gas (or vacuum) therein and are further designed to surround at least a portion of the antenna 72, thereby forming an enclosure that maintains the air gap proximal to the antenna. These tubes can be formed from glass or a variety of materials as described in more detail above. Although this exemplary embodiment illustrates one design wherein the tubes only surround a portion of the antenna, it can be advantageous in some embodiments to design the tubes 74 to fully surround the antenna 72 for example a U-shaped or C-shaped tubing structure to match a complementary-shaped antenna. In some alternative embodiments, other enclosures are contemplated that maintain an air gap proximal to and/or at least partially surrounding the antenna, for example, the electronics can be housed within a shell formed from a polymeric or other non-hermetic material, that forms the body of the sensor and wherein the antenna is located in a location spaced from the shell body. A variety of alternative configurations, such as described in more detail above, can be applied to the exemplary glucose sensor configuration. However, by providing the air gap between the antenna and material that forms the sensor body, consistent and reliable RF performance can be achieved even after implantation of the sensor in the body of a host.

While the systems and methods of the preferred embodiments are particularly well suited for use in conjunction with implantable glucose sensors, they can also be employed in any other implantable devices wherein neutral buoyancy, low dielectric constant, or some other characteristic feature is desirable, for example, pacemakers, sensors, and prostheses.

Methods and devices that are suitable for use in conjunction with aspects of the preferred embodiments are disclosed in co-pending U.S. patent application Ser. No. 10/885,476, filed Jul. 6, 2004, and entitled “SYSTEMS AND METHODS FOR MANUFACTURE OF AN ANALYTE SENSOR INCLUDING A MEMBRANE SYSTEM”; U.S. patent application Ser. No. 10/842,716, filed May 10, 2004, and entitled, “MEMBRANE SYSTEMS INCORPORATING BIOACTIVE AGENTS”; co-pending U.S. patent application Ser. No. 10/838,912, filed May 3, 2004, and entitled, “IMPLANTABLE ANALYTE SENSOR”; U.S. patent application Ser. No. 10/789,359, filed Feb. 26, 2004, and entitled, “INTEGRATED DELIVERY DEVICE FOR A CONTINUOUS GLUCOSE SENSOR”; U.S. application Ser. No. 10/685,636, filed Oct. 28, 2003, and entitled, “SILICONE COMPOSITION FOR MEMBRANE SYSTEM”; U.S. application Ser. No. 10/648,849, filed Aug. 22, 2003, and entitled, “SYSTEMS AND METHODS FOR REPLACING SIGNAL ARTIFACTS IN A GLUCOSE SENSOR DATA STREAM”; U.S. application Ser. No. 10/646,333, filed Aug. 22, 2003 entitled, “OPTIMIZED SENSOR GEOMETRY FOR AN IMPLANTABLE GLUCOSE SENSOR”; U.S. application Ser. No. 10/647,065, filed Aug. 22, 2003, entitled, “POROUS MEMBRANES FOR USE WITH IMPLANTABLE DEVICES”; U.S. application Ser. No. 10/633,367, filed Aug. 1, 2003, entitled, “SYSTEM AND METHODS FOR PROCESSING ANALYTE SENSOR DATA”; U.S. Pat. No. 6,702,857 entitled “MEMBRANE FOR USE WITH IMPLANTABLE DEVICES”; U.S. application Ser. No. 09/447,227, filed Nov. 22, 1999, and entitled “DEVICE AND METHOD FOR DETERMINING ANALYTE LEVELS”; and U.S. Publ. No. 2004-0011671 A1 entitled “DEVICE AND METHOD FOR DETERMINING ANALYTE LEVELS,” as well as published applications and issued patents including U.S. Publ. No. 2003/0217966 A1 entitled “TECHNIQUES TO IMPROVE POLYURETHANE MEMBRANES FOR IMPLANTABLE GLUCOSE SENSORS”; U.S. Publ. No. 2003/0032874 A1 entitled “SENSOR HEAD FOR USE WITH IMPLANTABLE DEVICE”; U.S. Pat. No. 6,741,877 entitled “DEVICE AND METHOD FOR DETERMINING ANALYTE LEVELS”; U.S. Pat. No. 6,558,321 entitled “SYSTEMS AND METHODS FOR REMOTE MONITORING AND MODULATION OF MEDICAL DEVICES”; U.S. Pat. No. 6,001,067 issued Dec. 14, 1999 and entitled “DEVICE AND METHOD FOR DETERMINING ANALYTE LEVELS”; U.S. Pat. No. 4,994,167 issued Feb. 19, 1991 and entitled “BIOLOGICAL FLUID MEASURING DEVICE”; and U.S. Pat. No. 4,757,022 filed Jul. 12, 1988 and entitled “BIOLOGICAL FLUID MEASURING DEVICE”; U.S. Appl. No. 60/489,615 filed Jul. 23, 2003 and entitled “ROLLED ELECTRODE ARRAY AND ITS METHOD FOR MANUFACTURE”; U.S. Appl. No. 60/490,010 filed Jul. 25, 2003 and entitled “INCREASING BIAS FOR OXYGEN PRODUCTION IN AN ELECTRODE ASSEMBLY”; U.S. Appl. No. 60/490,009 filed Jul. 25, 2003 and entitled “OXYGEN ENHANCING ENZYME MEMBRANE FOR ELECTROCHEMICAL SENSORS”; U.S. application Ser. No. 10/896,312 filed Jul. 21, 2004 and entitled “OXYGEN-GENERATING ELECTRODE FOR USE IN ELECTROCHEMICAL SENSORS”; U.S. application Ser. No. 10/896,637 filed Jul. 21, 2004 and entitled “ROLLED ELECTRODE ARRAY AND ITS METHOD FOR MANUFACTURE”; U.S. application Ser. No. 10/896,772 filed Jul. 21, 2004 and entitled “INCREASING BIAS FOR OXYGEN PRODUCTION IN AN ELECTRODE ASSEMBLY”; U.S. application Ser. No. 10/896,639 filed Jul. 21, 2004 and entitled “OXYGEN ENHANCING ENZYME MEMBRANE FOR ELECTROCHEMICAL SENSORS”; U.S. application Ser. No. 10/897,377 filed Jul. 21, 2004 and entitled “ELECTROCHEMICAL SENSORS INCLUDING ELECTRODE SYSTEMS WITH INCREASED OXYGEN GENERATION”. The foregoing patent applications and patents are hereby incorporated herein by reference in their entireties.

All references cited herein are incorporated herein by reference in their entireties. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

1. A device suitable for implantation in a body, the device comprising:
an antenna encapsulated within a non-hermetic material, wherein the antenna is spaced from the non-hermetic material by an air gap.

2. The device of claim 1, wherein the air gap is maintained by an enclosure.

3. The device of claim 2, wherein the enclosure surrounds at least a portion of the antenna.

4. The device of claim 2, wherein the antenna is contained within at least a portion of the enclosure.

5. The device of claim 2, wherein the enclosure comprises a hermetic material.

6. The device of claim 5, wherein the enclosure comprises glass.

7. The device of claim 2, wherein the enclosure comprises a non-hermetic material.

8. The device of claim 7, wherein the enclosure comprises a polymeric material.

9. The device of claim 2, wherein the enclosure comprises at least one tube.

10. The device of claim 9, wherein the tube comprises a wheel-like configuration.

11. The device of claim 1, wherein the air gap is maintained by at least one spacer that maintains a fixed distance between at least two support structures.

12. The device of claim 1, wherein the non-hermetic material comprises a plurality of hollow gas-filled beads.

13. The device of claim 1, wherein the device comprises a wholly implantable glucose sensor.

14. The device of claim 1, wherein the device comprises electronics, and wherein the non-hermetic material is molded around the electronics and antenna.

15. A method for forming a device suitable for implantation in a body, the method comprising:
providing device electronics comprising an antenna configured for radiating or receiving an RF transmission, wherein the antenna is at least partially surrounded by an enclosure; and
molding a non-hermetic material around the sensor electronics such that an air gap is maintained within the enclosure at least partially surrounding the antenna, whereby a device suitable for implantation is a body is obtained.

16. The method of claim 15, wherein the enclosure comprises a hermetic material.

17. The method of claim 16, wherein the enclosure comprises glass.

18. The method of claim 17, wherein the enclosure comprises at least one glass tube.

19. The method of claim 15, wherein the device comprises a wholly implantable glucose sensor, and wherein the electronics are configured to process a signal from the glucose sensor.
